(19) United States
(12) Patent Application Publication
(10) Pub. No.: US 2007/0010798 A1
(43) Pub. Date: Jan. 11, 2007

(54) Device and systems for the intermittent drainage of urine and other biological fluids

(75) Inventors: Marshall L. Stoller, San Francisco, CA (US); Maxwell V. Meng, San Francisco, CA (US)

Correspondence Address: BEYER WEAVER & THOMAS, LLP, P.O. BOX 70250, OAKLAND, CA, 94612-0250, US

(73) Assignee: The Regents of the University of California

(21) Appl. No.: 11/099,120

(22) Filed: Apr. 4, 2005

Related U.S. Application Data
(60) Provisional application No. 60/559,179, filed on Apr. 2, 2004.
(60) Provisional application No. 60/575,468, filed on May 27, 2004.

(51) Int. Cl. A61M 27/00

(52) U.S. Cl. 604/544; 604/320

(57) ABSTRACT

This invention provides a drainage device for biological fluids. The device typically comprises a port for attachment of the device to a catheter or to a waste collection device; and a means for applying a negative pressure to said port, wherein said negative pressure is an intermittent substantially constant negative pressure.

### CROSS-REFERENCE TO RELATED APPLICATIONS

This application claims benefit of and priority to U.S. Ser. No. 60/575,468, filed on May 27, 2004, and to U.S. Ser. No. 60/559,179, filed on Apr. 2, 2004, both of which are incorporated by reference in their entirety for all purposes.

### STATEMENT AS TO RIGHTS TO INVENTIONS MADE UNDER FEDERALLY SPONSORED RESEARCH AND DEVELOPMENT

Applicable

### FIELD OF THE INVENTION

This invention relates to collection devices and methods for use with urinary and other catheters.

### BACKGROUND OF THE INVENTION

A urinary drainage catheter, such as the Foley catheter, is a hollow, tubular device commonly used in the medical profession for insertion into a patient's bladder via the urethral tract to permit the drainage of urine. Use of a urinary catheter is often necessary for patients that are undergoing surgery, orthopedically incapacitated, incontinent, or incapable of voluntary urination. An unfortunate problem with catheterization, however, is the development of sepsis and/or urinary tract infections (UTIs) as a result of bacterial invasion in the bladder and urinary tract by various microorganisms. Sepsis is potentially lethal and most prevalent in the elderly, where urinary tract and bladder infections become systemic very easily, especially if hygiene is poor and hydration of tissue is deficient. The risk of sepsis increases with the employment of urinary drainage catheters, where normal flora, and/or bacteria from feces or skin easily ascend into the bladder around the inserted catheter.

In addition, residual urine in stasis around the retention balloon provides a culture medium at warm body temperatures that facilitates the growth of bacteria. Consequently, bacteria are able to accumulate, multiply and become pathogenic in the bladder, eventually circulating into the kidneys and throughout the system, resulting in sepsis of the system. Because of this propensity to produce infection in the patient, medical practitioners often refuse to extend the use of catheters, despite their usefulness.

### SUMMARY OF THE INVENTION

This invention provides a drainage device for biological fluids. The device typically comprises a port for attachment of the device to a catheter or to a waste collection device; and a means for applying a negative pressure to said port, wherein said negative pressure is an intermittent substantially constant negative pressure.

### DEFINITIONS

The term “intermittent” when used with respect to “negative pressure that is an intermittent substantially constant negative pressure” refers to a negative pressure that is applied for intervals and then discontinued.

The term “substantially constant”, when used with respect to a substantially constant negative pressure refers to a negative pressure that varies by no more than about 20%, preferably by no more than about 10% or 15%, and most preferably by no more than about 5%.

The term “closed system” refers to a system that can be manipulated by a user without substantial risk of contact with the biological fluids present in that system.

The term biological fluid refers to any one or more fluids produced by a biological organism. Such biological fluids include, but are not limited to urine, seminal fluid, cerebral spinal fluid, blood or blood fractions, plasma, saliva or other oral fluid, stomach fluid, bile, pus, liquefied tissues, and the like.

The term “catheter” is used herein to refer to any drainage tube for collection of a bodily fluid. Such devices include, but are not limited to a Foley catheter, any other catheter, a nasogastric tube, a Jackson Pratt Drain or bulb drain, and the like.

### BRIEF DESCRIPTION OF THE DRAWINGS

FIG. 1 schematically illustrates a drainage device 2 comprising an internal means 6 for generating a negative pressure.

FIG. 2 schematically illustrates a drainage device 2 for use with an external vacuum source 22

FIG. 3 schematically illustrates a drainage device 2 comprising a crank 20 for generating power for operation of the means 6 for generating negative pressure.

FIG. 4 illustrates one example of a “linear configuration” of the drainage/collection system according to this invention.

FIG. 5 illustrates one example of a “T-configuration” of the drainage/collection system according to this invention.

### DETAILED DESCRIPTION

This invention pertains to the surprising discovery that in most typical patients catheterized with an apparently free-flowing Foley catheter, substantial urine (e.g. 200-400 ml) remains in the bladder. This standing pool of urine increases the likelihood of infection (e.g., sepsis and/or UTIs) and reduces kidney drainage.

It was a surprising discovery that application of a low negative pressure (suction) to the catheter and/or to a receptacle into which the catheter drains, especially an intermittent (e.g., random, haphazard, or periodic) negative pressure effectively eliminates this residual pool of urine, reduces the onset of infection and promotes bladder and kidney health. In certain preferred embodiments the intermittent negative pressure, when applied, is a substantially constant negative pressure (e.g. the magnitude of the negative pressure varies by less than about 20 or 25 percent).

Without being bound to a particular theory, it is believed that the intermittent negative pressure eliminates “airlocks” in the catheter and associated waste/drainage system. It is also believed that the negative pressure overcomes surface tension effects that inhibit fluid flow through the catheter and/or waste/drainage system. In addition, it is believed that the intermittent negative pressure enhances a “siphon” effect in the catheter thereby enhancing evacuation of the bladder.

Moreover, it was also a surprising discovery that the intermittent quality of the negative pressure was more effective in this regard than a constant negative pressure. Without being bound to a particular theory, it is believed that a constant negative pressure results in the occlusion of the catheter by the mucosa (e.g. the mucosa lining the bladder) as the bladder fully evacuates. The intermittent negative pressure reduces or eliminates this occlusion and also reduces irritation to the mucosa.

While the systems of this invention are, in certain embodiments, particularly well suited to facilitate drainage of the bladder, it is believed that the methods and devices are also well suited to the drainage of other biological fluids (e.g., nasogastric fluids, intraperitoneal fluids, particularly from catheters inserted into infradiaphragmatic sites, and the like). Thus, while the devices and systems described herein are shown with reference to, e.g. a Foley catheter, it will be appreciated that they can be used similarly with essentially any device for collection of biological fluids. Such devices include, but are not limited to a nasogastric tube, a Jackson Pratt Drain or bulb drain, and the like.

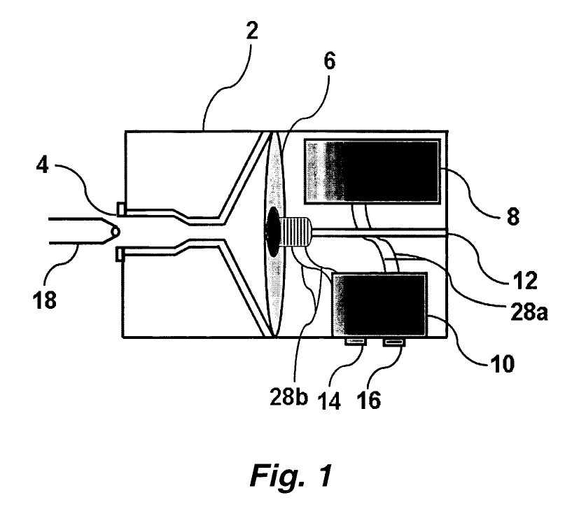

Thus, in certain embodiments, this invention provides a drainage device for biological fluids. One such device is illustrated schematically in FIG. 1. The device 2 typically comprises a port 4 for attachment of the device to a collection tube 18, catheter, or to a waste collection device; and a means 6 for applying a negative pressure to the port. The means for applying negative pressure in the embodiment illustrated in FIG. 1 is a diaphragm pump 6. The pump is connected, e.g. by wires 28b to a regulator 10 that in certain preferred embodiments provides a control 14 for regulating the on and/or off time of the pump, and/or a control 16 for regulating the magnitude of the negative pressure. The regulator and pump are powered by a power source 8 (e.g. permanent battery, removable battery, manual generator, battery/generator combination, etc.) connected to the regulator, e.g. through wires 28a. The negative pressure is typically an intermittent substantially constant negative pressure. While FIG. 1 illustrates a diaphragm pump, a wide variety of means for applying negative pressure are known to those of skill and will be suitable for use in this device. In the illustrated embodiment, the diaphragm pump 6 vents gas through port 12.

In certain embodiments, the means 6 for applying negative pressure comprises a vacuum pump that can be powered pneumatically, mechanically, or electrically. Suitable vacuum pumps include, but are not limited to a piston pump, a centrifugal pump, a bellows pump, a diaphragm pump, and the like.

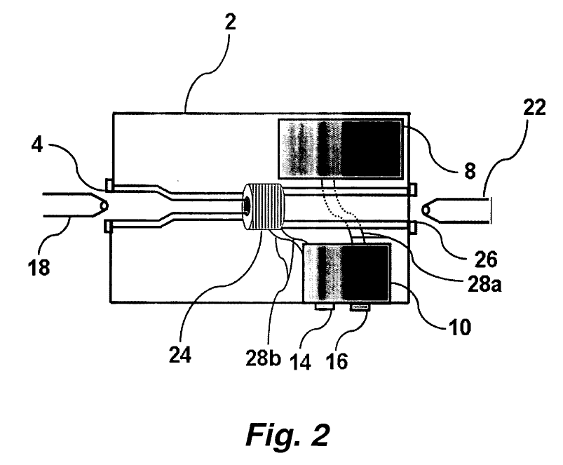

The devices of this invention are not limited to the use of various vacuum pumps. In certain embodiments, the devices can be configured for connection to an external vacuum source (e.g., wall vacuum). In such instances, illustrated schematically in FIG. 2, the device typically comprises a port 26 or other connector (nipple, flange, luer lock fitting, etc.) for connection to an external vacuum source 22. Rather than a pump, the device contains a pressure regulator/valve assembly 24. The regulator valve assembly regulates the magnitude of the negative pressure and cycles the valve to provide intermittent, substantially constant, negative pressure. The regulator valve assembly is connected, e.g. by wires 28b to a regulator 10 that, in certain preferred embodiments, provides a control 14 for regulating the on and/or off time of the valve, and/or a control 16 for regulating the magnitude of the negative pressure. In various embodiments, the regulator 10 derives power from the power source 8 through wires 28a as illustrated.

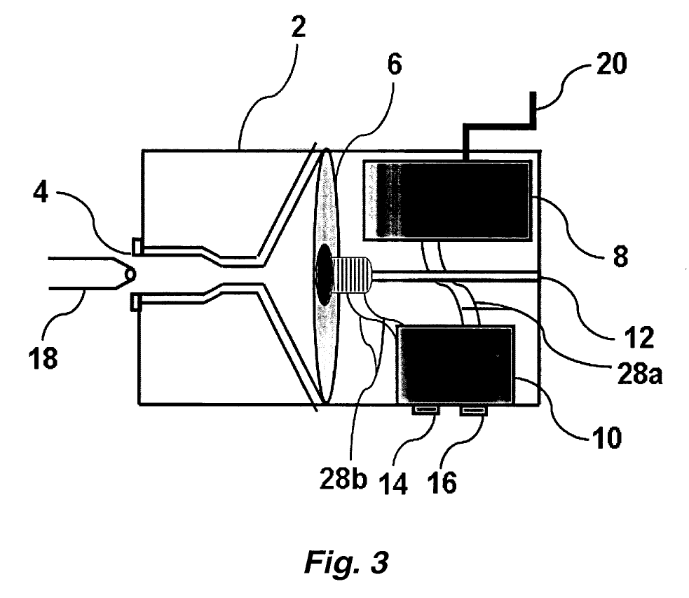

As indicated above, the device can be electrically powered or, in certain embodiments, manually powered. When manually powered, the means 6 for applying negative pressure can be a manually powered pump (e.g., a crank-driven pump, a “squeeze-bulb pump”, a manual piston pump, etc.) or an electrically-powered pump where the electrical power is ultimately derived from manual means (e.g. a crank 20/clockwork powered generator as illustrated in FIG. 3. Were the device is electrically powered, it can be powered by an external power source, and/or by an internal powered source (e.g. removable or permanently installed batteries). Certain embodiments contemplate a disposable device 2 comprising an integral battery. The battery can be rechargeable or when the battery is expired, the entire device 2 is replaced. Certain embodiments, contemplate a light-powered or light-recharged battery where the body of the device additionally comprises one or more photovoltaic cells to directly power the pump and/or to recharge a battery that powers the pump.

The pump, or other vacuum source, will typically be regulated to provide an intermittent suction and during the periods of suction, the magnitude of the negative pressure will be substantially constant (e.g., varying in magnitude by no more than about 20 or 25 percent).

In certain preferred embodiments, the device will be fully self-contained (including battery) and, optionally, disposable. The device can readily be affixed to a catheter (or other collection device) and/or to a waste receptacle that is attached to a catheter whereby the negative pressure is communicated to the lumen of the catheter.

In certain embodiments, the application of the negative pressure can be random, haphazard, or periodic. In certain embodiments, the period between applications of the negative pressure ranges from about every 30 seconds to about every four hours, preferably from about every minute to about every 1 or two hours, more preferably from about every 2 minutes to about every 30 minutes or one hour. The duration of application of the negative pressure typically ranges from about 5 seconds to about 1 hour, preferably from about 10 seconds to about 10 minutes, more preferably from about 10 seconds to about 30 seconds. In certain embodiments, the negative pressure is applied for about 15 seconds every 10 minutes.

The negative pressure is a relatively low negative pressure, in certain embodiments, ranging up to about 40 cm of water, preferably up to about 30 cm of water, more preferably from about 1 or 5 up to about 15 cm of water.

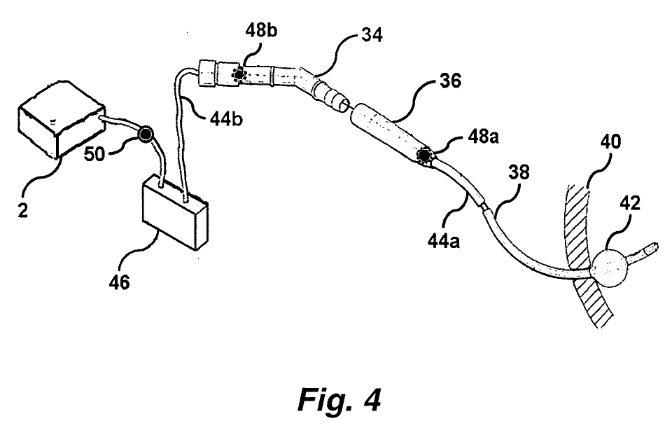

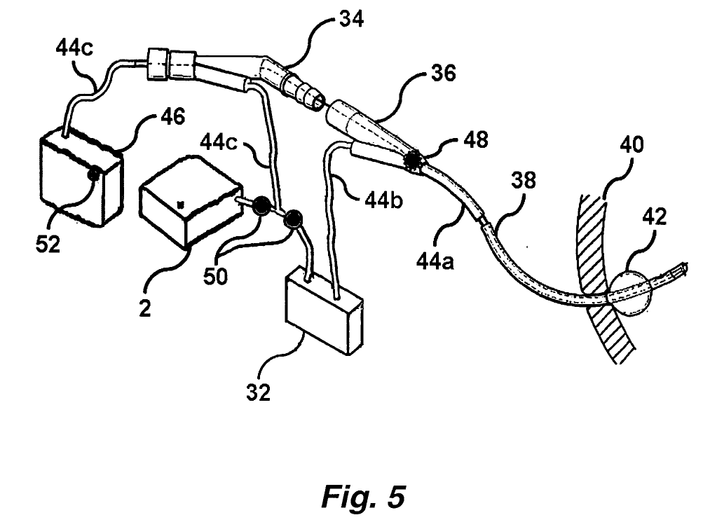

Various illustrative systems for effecting drainage of a bladder are illustrated schematically in FIG. 4, and FIG. 5. It is noted that while FIGS. 4 and 5 illustrate a Foley catheter, the systems can be similarly used with essentially any drainage device.

As illustrated in FIG. 4, the drainage device 2 can be used to drain a bladder 40. In the “linear configuration” illustrated in FIG. 4 a collection device/catheter 38 (illustrated as a Foley catheter comprising balloon 42) is coupled to a connecting tube 44a that terminates in a coupler 36. The coupler 36 mates with another coupler 34 that, in turn, is connected to waste collection device 46, e.g., a drainage bag, through a connecting tube 44b and/or 44c. The waste collection device 46 can, optionally comprise an anti-reflux trap that prevents backflow of urine (or other biological fluid) through the connecting tubes and/or drainage tube. The drainage bag can also optionally further comprise a trap that prevents flow of fluid into the drainage device (pump) 2.

When drainage device 2 is activated, a negative pressure is applied through the collection bag to the drainage tubes 44a, 44b to the collection device 38 (e.g. Foley catheter) and induces flow of fluid through the collection device and drainage tubes into the waste collection device 46. When the device 2 turns off the flow continues by means of a siphon effect. The system is typically equipped with a valve, e.g., on the waste receptacle 46 (e.g., drainage bag), between the waste collection receptacle 46 and the device 2, or as a component of the device 2. The valve, when open, allows air into or out of the system, and when closed isolated the system from the atmosphere. The valve (e.g., a ball valve, a flap valve, etc.) closes when device 2 is turned on allowing a negative pressure to form in the collection bag and/or the various tubes comprising the system thereby sucking fluid out of the biological site (e.g., the bladder). When the device 2 shuts off, the valve opens so that air can leave the system thereby permitting fluid to flow through the plumbing into the collection bag via a siphon effect. The system can be fitted with one or more check valves (e.g. 48a, 48b) at various locations to prevent backflow of fluid through the system. A check valve 50 can also be provided to prevent fluid flow into the device 2.

A “T-configuration” of the system is illustrated schematically in FIG. 5. In this configuration, the drainage device 2 is connected by means of a “T” or “Y” between the waste receptacle 46 and the collection device/catheter 38 (e.g. Foley catheter). In the embodiment illustrated in FIG. 5, the “T-” or “Y-” connection is effected by coupler 36, which permits the drainage device 2 and/or the waste receptacle 46 to be easily coupled or uncoupled from the system. The system is illustrated in FIG. 5 with an optional trap 32 that prevents fluid collected by the system from entering device 2. The system can optionally comprise check valve(s) 50 to also limit entry of fluid into device 2. In various embodiments, the valve(s) 50-are located at either or both of the indicated locations. The waste collection device 46 can, optionally comprise an anti-reflux trap that prevents backflow of urine (or other biological fluid) through the connecting tubes and/or drainage tube. In certain embodiments, the vaste collection device 46 comprises a one-way valve 52 when device 52 applies negative pressure to the system. When the negative pressure turns off, valve 52 opens allowing the waste receptacle to vent and permitting fluid to enter and fill that receptacle.

In various embodiments, the system is typically equipped with a valve, e.g., on the drainage bag, between the drainage bag and the device 2, etc. The valve, when open, allows air into or out of the system, and when closed isolated the system from the atmosphere. The valve (e.g., a ball valve, a flap valve, etc.) closes when device 2 is turned on allowing a negative pressure to form in the waste receptacle 46 and/or the various tubes comprising the system thereby sucking fluid out of the biological site (e.g., the bladder). When the device 2 shuts off, the valve opens so that air can leave the system thereby permitting fluid to flow through the plumbing into the waste receptacle via a siphon effect. The system can be fitted with one or more check valves (e.g. 48a, 48b) at various locations to prevent backflow of fluid through the system.

In certain embodiments, this invention also provides kits. In various embodiments, the kits typically include a drainage device 2 as described herein and a collecting means (e.g. a catheter or other collecting means) for application to the site. The kits are typically packaged so that the collecting means and/or the drainage device are sterile or can be placed in a conventional sterilizer. In certain embodiments, the kit further includes connecting tubing, and/or a waste receptacle for receiving biological fluid drained from the site. The kits can, optionally, further include instructional materials teaching the use of the drainage device with the collecting means. In various embodiments, the instructional materials further indicate appropriate negative pressures, and on and/or off durations for the drainage device for use in draining the site (e.g., a lung, an oral cavity, a thorax, a wound, a bladder, etc.).

While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

What is claimed is:

1. A drainage device for biological fluids, said device comprising:
a port for attachment of the device to a catheter or to a waste collection device;
a means for applying a negative pressure to said port, wherein said negative pressure is an intermittent substantially constant negative pressure.

2. The drainage device of claim 1, wherein said means for applying negative pressure is a manual pump.

3. The drainage device of claim 1, wherein said pump comprises a squeeze bulb.

4. The drainage device of claim 1, wherein said means for applying negative pressure is an electric pump.

5. The drainage device of claim 4, wherein said electric pump is battery powered.

6. The drainage device of claim 4, wherein said device further comprises a photovoltaic cell for powering said electric pump or for recharging a battery.

7. The drainage device of claim 4, wherein said electric pump is selected from the group consisting of a centrifugal pump, a bellows pump, a diaphragm pump, and a piston pump.

8. The drainage device of claim 1, wherein said device further comprises means for adjusting the period of said negative pressure and/or the magnitude of said negative pressure.

9. The drainage device of claim 1, wherein said device applies the negative pressure at an interval ranging from approximately every 30 seconds to approximately every 30 minutes

10. The drainage device of claim 1, wherein said device applies the negative pressure at an interval ranging from approximately every 30 seconds to approximately every 10 minutes.

11. The drainage device of claim 1, wherein said means for applying a negative pressure generates a negative pressure of up to about 100 cm of water.

12. The drainage device of claim 1, wherein said means for applying a negative pressure generates a negative pressure of up to about 40 cm of water.

13. The drainage device of claim 1, wherein said means for applying a negative pressure generates a negative pressure of up to about 15 cm of water.

14. The drainage device of claim 1, wherein said port is a port that attaches to a Foley catheter or a Foley catheter collection device.

15. The drainage device of claim 1, wherein said device is battery powered, the battery is an integral part of said device, and said device is disposable.

16. The drainage device of claim 1, wherein said device comprises an electric pump and electricity for said pump is provided by a manual generation system.

17. The drainage device of claim 16, wherein said manual generation system is a wind-up clockwork generator.

18. A system for effecting drainage of a bladder, said system comprising:
a catheter for insertion into said bladder;
a drainage device according to any of claims 1 through 17; and
a waste collection device in fluid communication with said catheter.

19. The system of claim 18, wherein said catheter is a Foley catheter.

20. The system of claim 18, wherein said catheter is a Jackson Pratt tube.

21. The system of claim 18, wherein said waste collection device is a bedside drainage bag.

22. The system of claim 21, wherein said waste collection device comprises an anti-reflux trap.

23. The system of claim 18, wherein said system further comprises a trap to prevent back flow of fluid into said drainage device.

24. The system of claim 23, wherein said trap is a component of the waste collection device.

25. The system of claim 23, wherein said trap is separate or separable from said waste collection device.

26. The system of claim 18, wherein said system further comprises one or more check valves to prevent backflow of fluid through said system.

27. The system of claim 18, wherein said system further comprises-a vent valve to close the system from atmosphere when the drainage device is applying a negative pressure and to open the system to atmosphere to allow outflow of air as fluid moves through the system into the waste collection device.

28. The system of claim 18, wherein said system is a closed system.

29. The system of claim 18, wherein said drainage device is connected in a T-configuration between said catheter and said waste collection device.

30. The system of claim 18, wherein said drainage device is connected to said waste collection device in a linear configuration.

31. A method of reducing urinary tract infection in a subject bearing a urinary catheter, said method comprising:
applying to said catheter an intermittent substantially constant negative pressure, whereby said negative pressure reduces urine retention in the bladder of said subject thereby reducing the incidence of urinary tract infection.

32. The method of claim 31, wherein said applying comprises using a device according to any of claims 1 through 17.

33. The method of claim 31, wherein said negative pressure is provided by a suction device affixed to said catheter or to collection tubing or to collection bag.

34. The method of claim 31, wherein said negative pressure is provided by a suction device affixed to a waste receptacle affixed to said catheter.

35. The method of claim 31, wherein said intermittent substantially constant negative pressure is applied at an interval ranging from approximately every 30 seconds to approximately every 10 minutes.

36. A method of draining a biological fluid from a site in a subject, said method comprising:
providing a collecting means at said site in said subject;
placing a device according to any of claims 1 through 17 in fluid communication to said collecting means so that when said device is activated flow is induced through said collecting means into a waste receptacle.

37. The method of claim 36, wherein said collecting means is selected from the group consisting of a Foley catheter, a nasogastric tube, and a Jackson Pratt tube.

38. The method of claim 36, wherein said site is selected from the group consisting of a lung, an oral cavity, a thorax, a wound, and a bladder.

39. A kit for draining a biological fluid from a site in a subject, said kit comprising:
a collecting means for application to said site; and
a drainage device according to any of claims 1 through 17.

40. The kit of claim 39, further comprising a waste receptacle for receiving biological fluid drained from said site.

41. The kit of claim 39, wherein said collecting means is a catheter.

42. The kit of claim 39, wherein said collecting means is selected from the group consisting of a Foley catheter, a Jackson Pratt tube, and a nasogastric tube.

43. The kit of claim 39, wherein said kit further comprises instructional materials teaching the use of said drainage device with said collecting means.

44. The kit of claim 43, wherein said instructional materials further indicate appropriate negative pressures, and on and/or off durations for said drainage device for use in draining said site.

45. The kit of claim 39, wherein said site is selected from the group consisting of a lung, an oral cavity, a thorax, a wound, and a bladder.
